(19) United States
(12) Patent Application Publication
(10) Pub. No.: US 2016/0374682 A1
(43) Pub. Date: Dec. 29, 2016

(54) SURGICAL PROCEDURE AND DEVICES FOR USE THEREIN

(71) Applicant: BALATECH LIMITED, Camterbury Kent (GB)

(72) Inventors: Paul Anthony LEONARD, London (GB); Duncan Dalgleish JACKSON, Chicago, IL (US); Kenneth PRENDERGAST, London (GB); Eoin BILLINGS, London (GB)

(21) Appl. No.: 15/125,154

(22) PCT Filed: Mar. 10, 2015
(86) PCT No.: PCT/EP2015/054989

§ 371 Date: Sep. 10, 2016

(30) Foreign Application Priority Data
Mar. 10, 2014 (GB) 1404214.7
Aug. 11, 2014 (GB) 1414229.3

(51) Int. Cl. A61B 17/11; A61F 2/856; A61F 2/06; A61F 2/958; A61F 2/844

(52) U.S. Cl. CPC A61B 17/11; A61F 2/958; A61F 2/844; A61F 2/064; A61F 2/856; A61B 2017/1107; A61B 2017/1139; A61F 2210/0004; A61F 2220/0008

(57) ABSTRACT

The creation of arteriovenous fistulas in renal dialysis patients is associated with various problems. By placing a valve which can control the blood flow between artery and vein, material improvements in renal dialysis patient care can be achieved. In a preferred embodiment, the valve is mounted on a ring located around an aperture in the side of an asymmetrically expanded stent set in the artery. Novel stents, valve structures and valve ring insertion arrangements are also described.

### FIELD OF THE INVENTION

This invention relates to a new surgical procedure, devices adapted for use in it, and implantable novel stents and valves for use in vascular surgery.

### BACKGROUND TO THE INVENTION

In the following discussion of the background to the invention, reference is made by way of numbered superscripts to various published documents or disclosures. These are as follows:
1 Fresenius Annual Report (ESRD Patients in 2011, A Global Perspective)
2 Am J Kidney Dis. 2006,48 (Supplemental) S176-S247
3 Patel S T, Hughes J, Mills J L, J Vasc. Surg. 2003; 38(3):439-445. discussion 445.
4 Beathard G A, Arnold P, Jackson J, Litchfield T, Kidney Int. 2003; 64(4):1487-1494.
5 Tordoir J H, Rooyens P, Dammers R, der Sande F M van, de Haan M, Yo T I. Nephrol Dial Transplant. 2003; 18(2):378-383.
6 Spergel L M, Ravani P, Roy-Chaudhury P, Asif A, Besarab A. J Nephrol. 2007; 20(4):388-398.
7 Roy-Chaudhury P, Spergel L M, Besarab A, Asif A, Ravani P. J. Nephrol. 2007; 20(2):150-163.
8 Robbin M L, Chamberlain N E, Lockhart M E, et al. Radiology. 2002; 225(1):59-64.
9 Falk A. Maintenance and salvage of arteriovenous fistulas. J Vasc Intery Radial. 2006; 17(5):807-813.
10 Silva M B Jr, Hobson R W II, Pappas P J, et al. J Vasc Surg 1998; 27:302-308.
11 Lomonte C, Casuci F, Antonelli M, et al. Semin. Dial. 2005; 18: 243-6.
12 S. Shenoy, J. Vasc. Access 2009; 10,223-232.
13 Ferring M, Henderson J, Wilmink T, J Vasc Access. 2014 July; 15(4):291-7.
14 Am. Journal of Kidney Disease 2001; 37 (Suppl. 1): S-7-S64

The treatment of patients with end stage renal disease (ESRD) is a growing problem: one which is straining the resources of healthcare providers around the world as well as seriously compromising the quality of life for the patients concerned. Globally, 148 countries are recorded as providing dialysis care to nearly 3 million patients with renal failure, approximately 2 million of whom receive haemodialysis (rather than peritoneal dialysis or kidney transplant). Global patient numbers have steadily risen at between 6% and 7% annually, significantly higher than the population as a whole.¹

More than half of the global dialysis patient population is accounted for in just 5 countries; the USA, Japan, China, Brazil and Germany. The USA alone accounts for approximately 20% of the global patient population. Importantly, in the context of the novel device presented here, there is now universal agreement that the preferred method of providing blood access for dialysis patients is through the creation of an arteriovenous fistula, rather than the use of shunts/grafts or catheters (which have been widely used to date, especially in the US). An arteriovenous fistula is essentially an aperture between an artery and a vein which allows blood to flow from the artery into the vein so as to increase the venous blood flow to a degree sufficient to allow for effective dialysis.

Optimising Arteriovenous Fistula Maturation and Preventing Primary Failure

Autogenous arteriovenous fistulas (AVFs) are recognised as the most attractive option for blood access for haemodialysis patients. Grafts and tunnelled catheters are more susceptible to infection and thrombosis and thus more likely to encounter complications and fail.²

Since 2003, the Fistula First initiative in the US has sought to increase the use of AVFs to account for the vast majority of blood access options for dialysis patients. It is clear that for such an initiative to succeed, and to achieve the best outcome for patients, increased fistula placement, successful maturation and enhanced long-term patency are all crucial factors.

Approximately one quarter to one third of primary fistulas never mature. Although much work has been done to improve vessel selection through ultrasound and other venography methods, fistula maturation rates remain relatively unchanged.^{3,4,5}

Successful fistula maturation depends upon:
Increased venous blood flow
Increased vein diameter
Increased visibility/accessibility of the vein
Easy cannulation within 90 days
Adequate blood flow to support dialysis

According to the US National Kidney Foundation's Kidney Disease Outcomes Quality Initiative (K/DOQI) an adequate fistula resides about 0.6 cm under the skin, has a flow rate of greater than 600 ml/min, and a diameter of about 0.6 cm.² However, these performance criteria and dimensions will change depending upon the body mass, general health and dimensions of the patient. These are just guidelines: other studies suggest that a vein diameter of greater than 0.4 cm and flow rate greater than 500 ml/min are good predictors of a working fistula likely to reach maturation.⁶ Recent studies also suggest that an end-diastolic arterial velocity of greater than 110 cm/s was also a good indicator of suitability for dialysis use.¹³ In effect, the fistula must deliver a blood flow rate in the vein sufficient to achieve a Kt/V value 1.2 (where “K” is the clearance of urea by the dialyser, “t” is the time taken for dialysis, usually about 3 hrs, and “V” is the patent's total fluid volume). To achieve effective dialysis, flow rates through the dialyser can vary from about 200 ml/min to greater than 400 ml/min: higher flow rates usually result in more efficient dialysis treatment.¹⁴

Roy-Chaudhury et al⁷ provides a comprehensive summary of the most significant factors governing primary (early) fistula failure. These include; shear stress, increases in transmural pressure, genetic predisposition, compliance disparities between the vein and the artery, turbulence, injury to the mobilised segment (causing hyperplasia) and thrombosis. This latter is usually due to technical errors and errors of judgement regarding vessel size/functionality, or other factors such as hypotension.⁶

Other important factors influencing increasing the likelihood of fistula failure include; the presence of diabetes, the location of the first fistula created, and the surgical expertise of the practitioner.⁸ Stenotic lesions are, further, a significant cause of early fistula failure.⁹

On the creation of a fistula, the arterial flow increases significantly as the body's response to maintain adequate perfusion pressure in the capillary beds distal to the anastomosis (to combat Steal Syndrome—the feelings of coldness in the hand (in the case of the preferred fistula site, between the radial artery and the cephalic vein in the lower arm), tingling and discomfort, anoxia, ischaemia and polyneuropathy). This, along with the effect of multiple needle sticks in the patient's vein may be the cause of venous thickening and hyperplasia, rather than swelling and effective fistula maturation.¹² Narrowing of the outflow vein is a prime cause of a fistula failing to mature properly, or at all.

Fistula/Vessel Site Selection

The K/DOQI guidelines² indicate an order of preference for fistula site creation, based upon ease of fistula access, quality of performance (achievable flow rates), likelihood of infection and failure, and the likelihood of complications due to Steal Syndrome. Veins selected for fistula creation usually require luminal diameters greater than 0.25 cm, and arteries greater than 0.2 cm, though there may be variations in luminal diameters based upon clinical factors or surgical preference.¹⁰ Healthy children, for example, can use arteries smaller than this which nevertheless produce adequate inflow to the fistula. Veins with luminal diameters of greater than 0.4 cm are generally required for grafts. It is generally the case that the non-dominant arm, (and the forearm rather than upper arm) is preferable, although a dominant forearm is preferable to a non-dominant upper arm. An upper extremity AVF is preferred to a graft and, in turn, a graft is preferred to a catheter. The primary reason is the likelihood of infection, and also the fact that catheter flow rates are often low.

From most to least desirable, the major blood access sites are set out as set out below.
A forearm cephalic vein AVF (radial artery-cephalic vein), followed by an upper arm cephalic vein AVF (brachial artery-cephalic vein), is preferred.
If it is not possible to create either of these fistulae, access may be established using a transposed basilic vein fistula (brachial artery-basilic vein), or other AVF configuration.
If the vascular anatomy is not suitable for any AVF placement, a graft of synthetic material (e.g. polytetrafluoroethylene [PTFE]) may be placed. A forearm loop graft (brachial artery to antecubital vein) is preferred over an upper arm straight graft (brachial artery to basilic vein). If no other upper extremity access is possible, an upper arm loop graft (axillary artery to axillary vein) may be placed if the anatomy is suitable.
Thigh grafts (superficial femoral artery to great saphenous vein or common femoral vein) are the next usual site for access placement.

Depending on patient anatomy and clinical considerations, other sites/vessels may be used but they will generally be less favourable and more problematic than the primary points of access listed above.

Site selection is important for predicting patency rates. For primary fistulas, current patency rates vary considerably, but they are generally patent for approximately 3 years. Secondary patency rates are highly dependent upon where the fistula is created: about 7 years for the forearm, and less than half that duration (3-3.5 years) for upper arm fistulas.

Complications and Counter Indications of AV Fistula Creation

Most complications of associated with fistula creation affect the fistula function directly; some of them are an immediate vital risk. Complications include:
Thrombosis/Stasis/Clotting/Stenosis
Bleeding and haematoma
Anastomotic pseudoaneurism
Venous aneurism
Venous pseudoaneurism
Skin necrosis (around the access site)
Ischaemia/neuropathy (Steal Syndrome)
Hyperdynamic syndrome/cardiomyopathy/ventricular hyperplasy
Edema of the extremities
Hyper/hypotension
Lymphorrhea
Infection

The primary cause of the above complications (in whole or in part) is the diversion of the natural blood flow and associated pressure imbalances mediated by the creation of the fistula.

### SUMMARY OF THE INVENTION

### Detailed Disclosure and Exemplary Embodiments

The invention concerns a surgical procedure and novel implant designed to facilitate the creation of an arteriovenous fistula, reduce the likelihood of primary fistula failure, and provide control of blood flow through the fistula to minimise the counter-indications described above.

In accordance with a first feature of the present invention, this specification discloses a surgical procedure which provides for a fistula to be created between an artery and a vein where the flow of blood though the fistula can be regulated and the fistula aperture can be reliably maintained.

More specifically, the invention in a first embodiment provides a surgical procedure for establishing an arteriovenous fistula between an artery and a vein enabling blood to flow directly from the artery into the vein wherein apertures in the walls of artery and vein are made, and the edges of the apertures are fixed to a valve member which can be selectively opened or closed to increase or decrease or cut off blood flow from the artery into the vein.

In a second feature, the specification discloses an arterial implant consisting of a novel stent structure, which is adapted and constructed to provide:
The ability to be securely anchored and cope with shear stress;
Predictable anastomosis and blood flow through the fistula;
Minimisation of turbulence through the fistula (or pooling);
Biocompatibility.
The capability of being employed in side to side or end to side fistulas.

The stent may also have drug-eluting capability and may also be equipped with sensor technology to monitor blood flow through the fistula.

Specifically, the invention provides a stent structure for use in surgery, the structure comprising an expandable stent body having an inlet and an outlet end and characterised by an aperture in the wall of the stent intermediate its ends and adapted to enable liquid flowing through the stent to pass from or into the vein or artery in which the stent is deployed.

Such a stent is placed in the artery (or possibly the vein) so that an aperture of a predetermined size can be made and maintained between the artery and the vein in an arteriovenous fistula. Depending upon the internal diameters of the artery and the vein, and the blood pressures therein, the stent allows for an aperture of a size sufficient for effective dialysis while minimising the Steal Syndrome effect and minimising the haemodynamic burden on the heart while allowing sufficient blood flow to achieve a Kt/V value of approximately 1.2, and hence afford effective dialysis.

The stent is preferably similar to a coffin in shape, wider at the top than at the bottom, and expanding outwards at the point of the aperture. The aperture itself is positioned towards the top of the stent. The wider top structure and shoulders leading to the aperture allow a slight drop in blood velocity above the aperture minimising shear stresses between the artery wall (endothelium) and the stent. This reduces the risk of bacterial build-up or other unwanted debris getting between the stent and the endothelium (which can lead to stenosis), as well as reducing turbulence and shear stress from blood flow though the aperture. The stent extends further downstream than upstream, again, to minimise the shear stress on the structure as a whole.

Maintaining a predetermined, optimal fistula anastomosis, such a device promotes effective fistula maturation (combating venous thinning or collapse). It also minimises the Steal Syndrome effect since the diverted blood from the artery would be just sufficient for dialysis and no more and, similarly, it would minimise the haemodynamic burden and the likelihood of cardiomyopathy.

Such a stent may be coated with a chemical substance (e.g. polyphosphazine) which allows for the elution of anti-stenosis, anti-thrombosis and/or anti-clotting agents.

Such a stent can be made from a number of biocompatible materials, including Nitinol (an alloy of Nickel and Titanium usually in the ratio 55% Nickel to 45% Titanium), or other shape memory alloy; Gold, Titanium, Cobalt-Chromium alloy or Tantalum. Or it might be of a suitable synthetic, polymeric material (silicone, polyethylene, polyurethane etc.) and/or shape memory polymers (SMPs) or a mixture of polymers whereby such polymers may be thermally-induced, light-induced, electromagnetically induced or electroactive and may have two or three transition states.

Such a stent may also comprise a sensor (based upon Doppler or piezoresistive technology) to allow for constant, real-time monitoring of fistula function. This would allow the patient to monitor his/her own fistula function (through a smart phone app, for example) or even allow practitioners to monitor their patients' fistula function remotely from the dialysis centre.

In a third feature, the invention provides an arteriovenous implant consisting of an actuatable valve mechanism within a novel stent structure, or as an integral part of a stent structure, which can be opened to enable blood flow from the artery to the vein and closed when no such flow is desired, or partially closed when a reduced flow rate is desired. Specifically, the invention provides a valve unit for use in the control of blood flow through an arteriovenous fistula which comprises a ring of biocompatible material adapted to be attached at its periphery to incisions made in an artery and a vein and, located bridging the ring, a flow restricting or flow occluding member which may be actuated to vary the size of the aperture in the ring.

Such an implant is placed directly between the vein and the artery, usually secured by a stenting structure in the artery (possibly internal or external) and possibly in the vein. The implant contains an aperture which may be selectively opened and closed or partially closed and which will, once the implant is in place, normally remain closed or partially closed. The aperture can, however, be opened during a dialysis session.

A wide variety of structures is conceivable for enabling the opening or closing of the valve aperture in the disclosed implants. One such is that the aperture may be closed by a flap including magnetic material or a permanent magnet which may be moved by the appropriate positioning of a magnet external to the patient to open up the aperture and permit flow through it.

Alternatively, using appropriate material, it is possible to design a structure which, when subjected to external pressure, opens up the aperture and, when the pressure is released, allows it to close again. A similar design might employ a “snap-open-snap-shut” mechanism. A demand valve or iris-type aperture might also be suitable. Piezoelectric, electromagnetic (solenoid-actuated), electrostatic or electrorotatory valves might also be employed.

One conceivable embodiment of the valve could utilise an electroactive polymer material which stiffened or relaxed, expanded or contracted under the influence of a radiofrequency or electromagnetic pulse, the valve being closed when stiffened or expanded and open when relaxed or contracted. Shape memory polymers which can assume distinct shapes and orientations and be thermally-induced, light-induced, or electrically/electromagnetically-induced might also be employed.

Similarly, carbon nanotube microfibers (aerogels) which can act as artificial muscles and can be made to constrict under an applied voltage and when housed within a suitable flexible and biocompatible material (silicone, or any other suitably pliant and biocompatible material) can form a valve which works in a similar fashion to a natural sphincter. Electrically contractible polymeric materials are also known which have the same mechanical behaviour as carbon nanotubes, and such materials could also be used.

Other designs of valve may be used provided they can be actuated during dialysis, can be securely located in place, and do not cause too much turbulence in the blood flow from artery to vein which could lead to thrombosis, haemolysis, stenosis or other complications. The default valve position should be open so that, should the valve fail, blood access is not compromised.

It will be appreciated that, once such an implant is fitted, normal, or near normal blood pressure and flow is maintained for around 95% of the time. This will prevent or reduce the likelihood of Steal Syndrome and will ameliorate the cardiac complications described above. Patients with upper arm fistulas (e.g. brachiobasilic and brachiocephalic fistulae), which are particularly prone to causing steal syndrome, will greatly benefit from the valve (which may also be used with a shunt should a shunt be necessary). Such an implant has the potential to make fistulas in the elbow or upper arm equally desirable as fistulas in the wrist, greatly widening the site options available for patients with problematic vascular anatomy or other complicating factors.

When the patient is to be dialysed, the valve is opened and blood flows through the fistula and into the vein. This causes the vein to swell, due to the increased pressure, and thus makes it easier for the needles to be inserted. After dialysis has taken place, the valve is closed, or partially so, preferably before the needles are removed so that removal is not carried out under excess pressure. This will ameliorate problems associated with venous, pressure-related damage, promote healing and reduce the likelihood of infection and/or excess thickening of the vein wall. The use of such a valve will depend upon whether the fistula is side (of the artery) to side (of the vein), or side (of the artery) to end (of the vein). In the former case, it might be desirable to fully close the valve while the patient is not undergoing dialysis, allowing normal venous pressure to persist in the vein. This may not be desirable since some additional pressure may be required to maintain sufficient blood flow in the inflated vein to prevent pooling, clotting and thrombosis. In which case, the valve will remain in a partially open state to allow for adequate venous flow. In the case of a side to end configuration, this will certainly be the case.

It will be appreciated that on creation of the fistula, the valve may be inserted in the fully closed position so that there is no blood flow though the anastomosis while the artery and vein heal and knit together post surgery. Only after healing is complete will the valve be opened to render the fistula operational. By allowing healing to take place in a non-turbulent environment where normal blood flow persists the likelihood of successful fistula maturation is increased. A fully controllable valve would allow for the vein to swell and develop in controlled conditions, to attain the appropriate size necessary to achieve the desired flow rate, and a Kt/V value of about 1.2.

It is also conceivable that an implant of the type discussed above may include a sensor to detect and measure blood flow during dialysis, possibly in combination with a suitable apparatus, which might be a suitably programmed smartphone, located on or near the patient's skin in the area of the fistula. The capacity to emit far infra-red light as an anti-stenosis measure is also conceivable. Further, such an actuatable valve could be used to address incontinence as a replacement urinary or anal sphincter, or other defective sphincter in the body, such as the iliocaecal valve.

The invention further provides an application and insertion tool for use in the procedure. Specifically, the invention provides an assembly of a mounting ring and insertion tool for use in locating a mounting ring in an aperture wherein the mounting ring has a plurality of outwardly facing slots and the insertion tool has a pair of jaws with ends engageable in the slots to hold the ring between them, and a slidable sleeve set around the jaws and having an internal shape corresponding to the shape of the ring and movable to a position in which the ring is held wholly within the sleeve, and wherein the mounting ring has a plurality of elastic members attached to its periphery which may be elastically deformed to enable the ring to be inserted in the sleeve and which, when the ring is released from the sleeve, spring out to extend beyond the periphery of the ring.

The invention is further explained in what follows with reference to the accompanying drawings, in which:

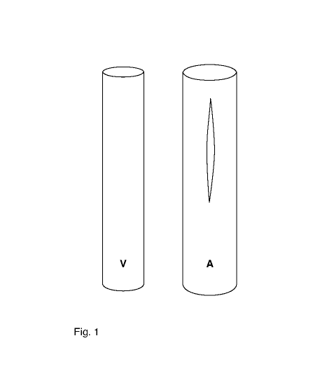

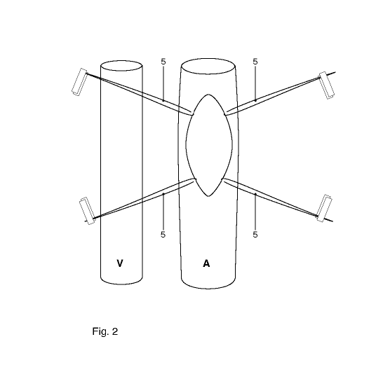

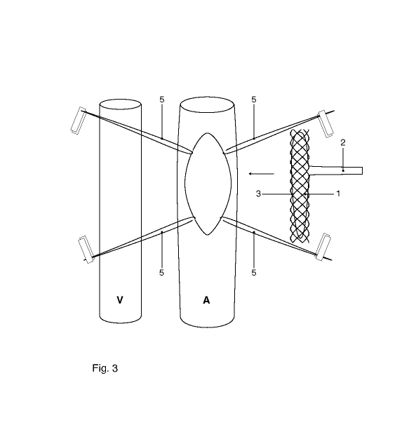

FIGS. 1 to 6 show diagrammatically the process of forming an arteriovenous fistula in accordance with the present invention;

FIGS. 6A.1 to 6A.13 show diagrammatically an alternative procedure for forming the fistula;

FIGS. 6B.1 to 6B.8 show a further alternative procedure;

FIG. 7 shows diagrammatically three possible forms of stent structure according to the invention;

FIG. 8 shows diagrammatically a valve insert member;

FIGS. 8A and 8B show diagrammatically how the valve member of FIG. 8 may be deployed in practice;

FIGS. 9 to 17 show diagrammatically a variety of stent/valve structures in situ in a patient; and

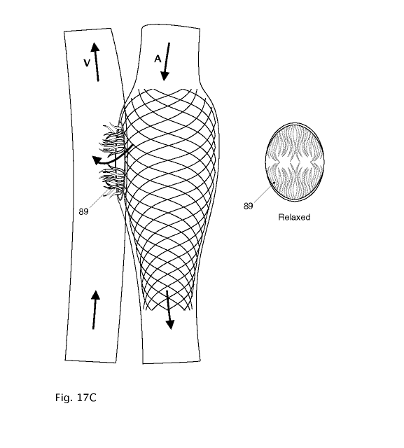

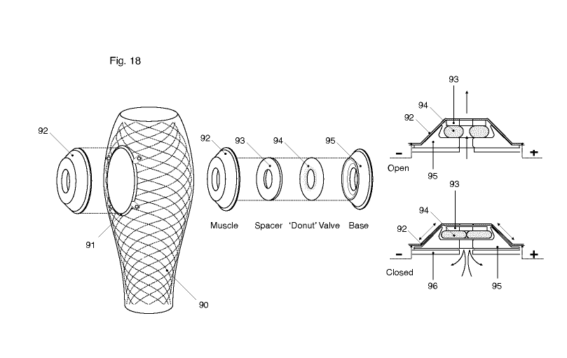

FIG. 18 shows diagrammatically an electrically actuated valve structure which may be used to control the fistula.

The following description of a surgical procedure, insertion tools, novel support ring and techniques for anastomosis creation makes reference to FIGS. 1 to 6. In this and all other relevant figures, the artery and vein between which the fistula is to be created are marked A and V.

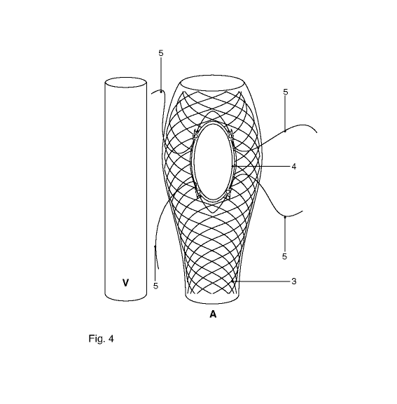

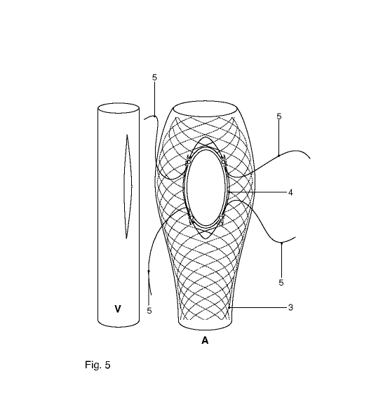

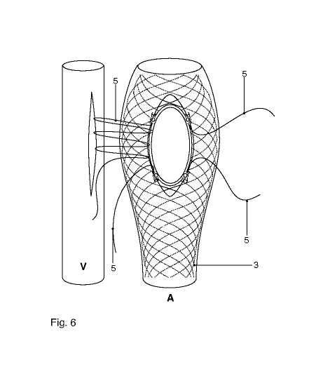

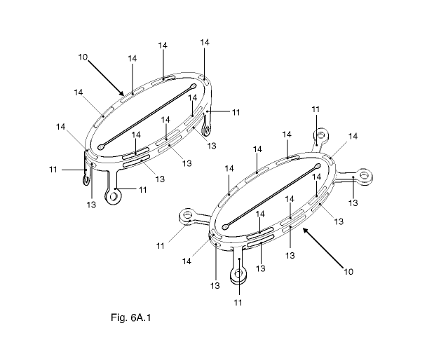

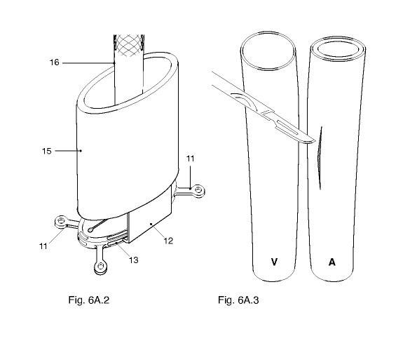

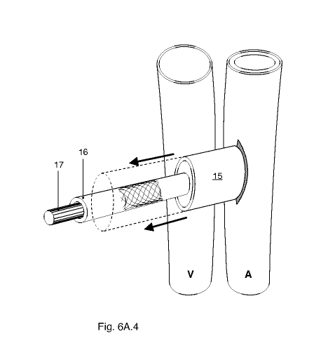

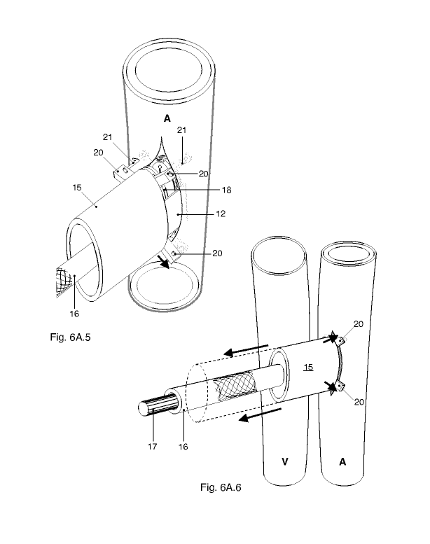

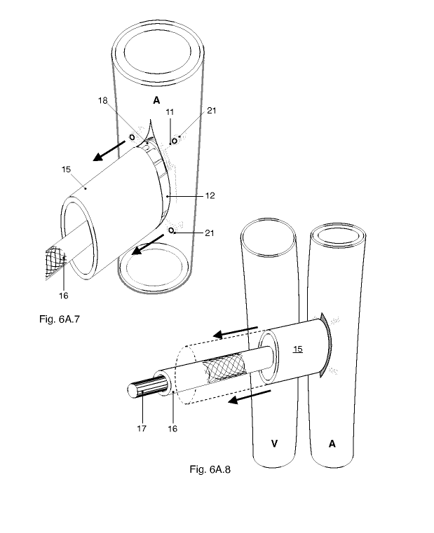

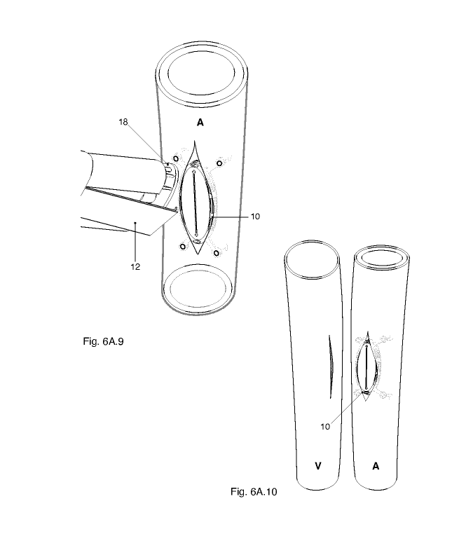

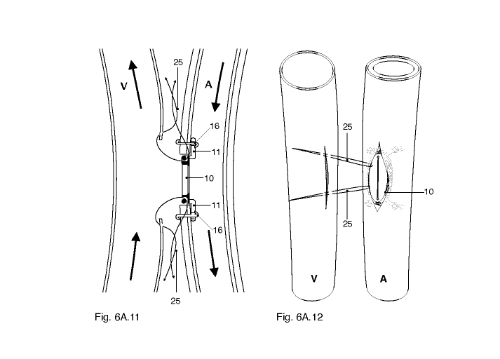

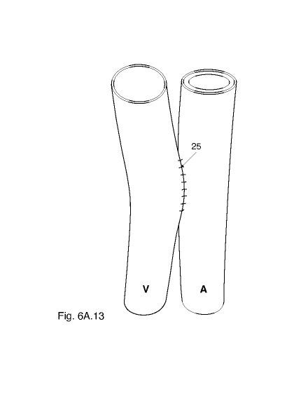

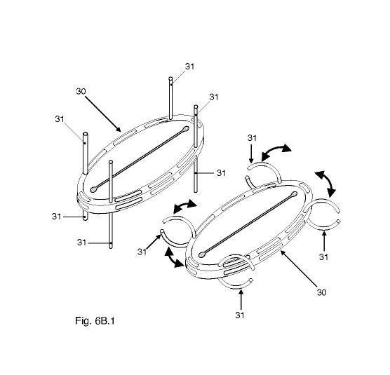

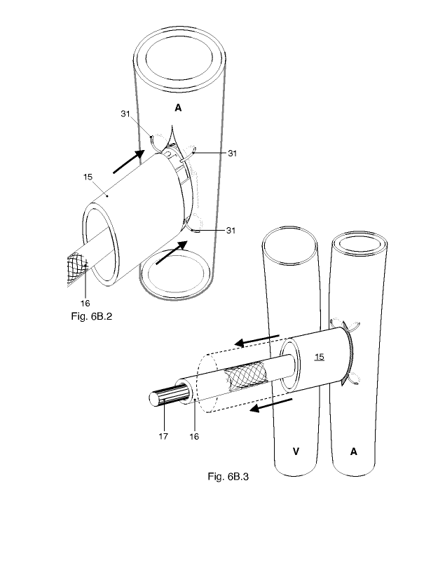

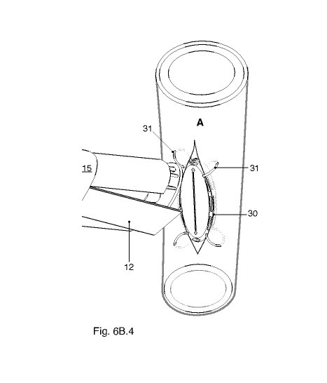

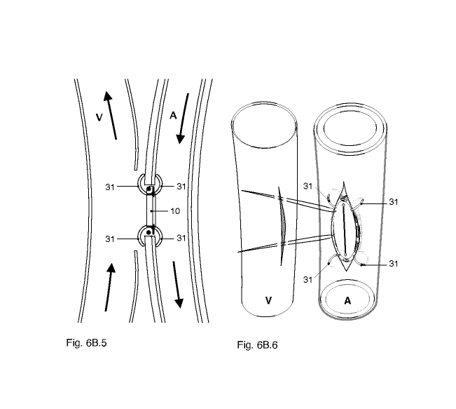

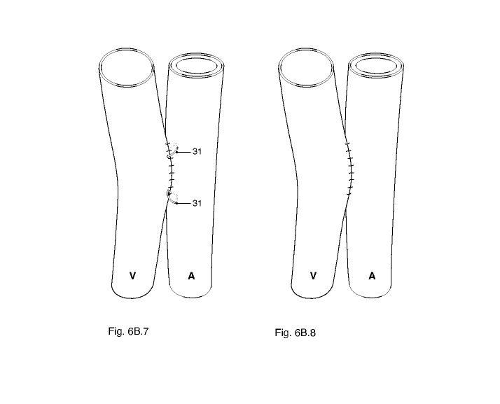

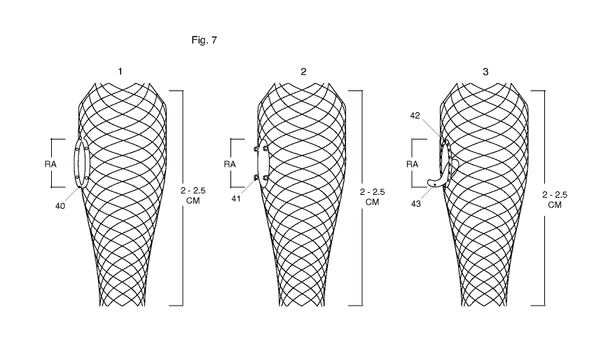

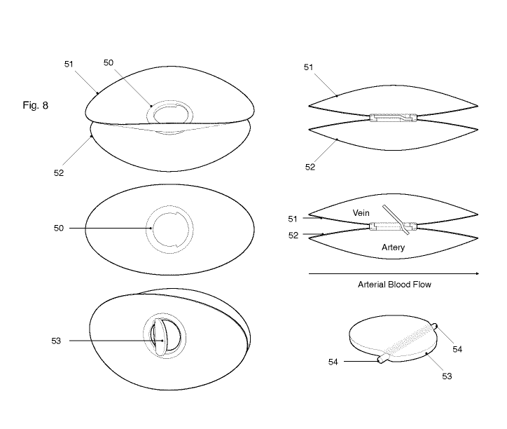

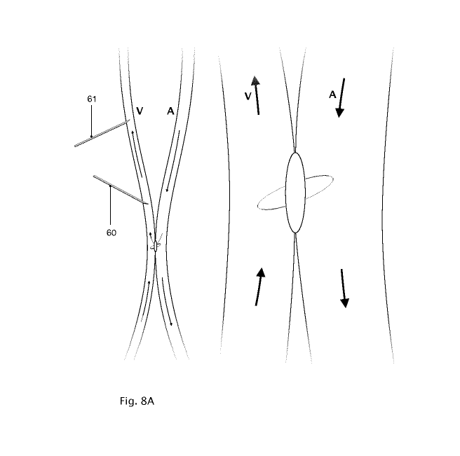

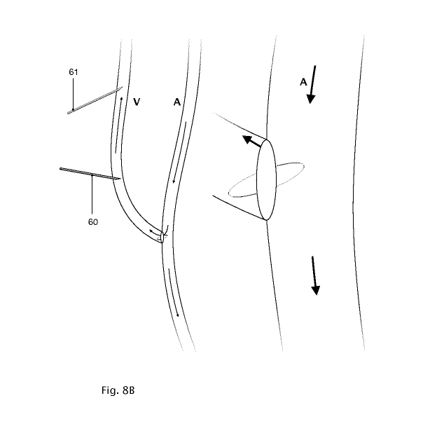

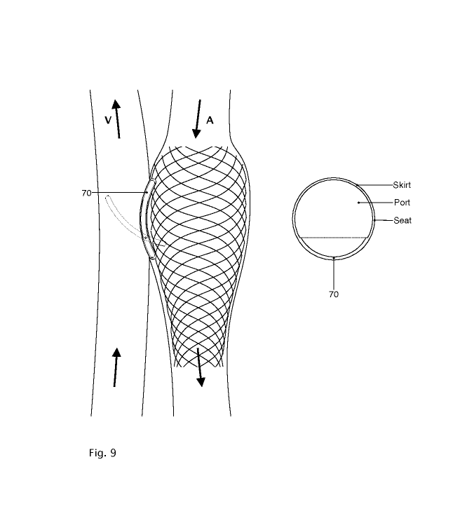

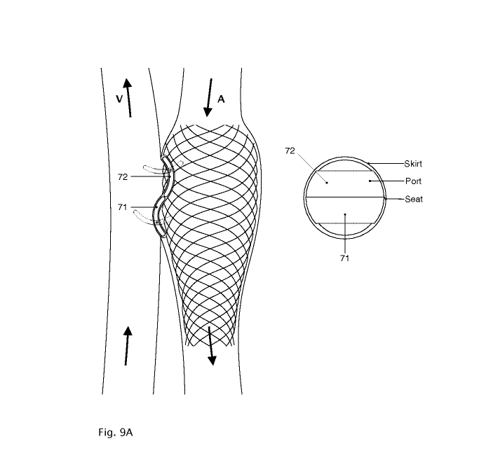

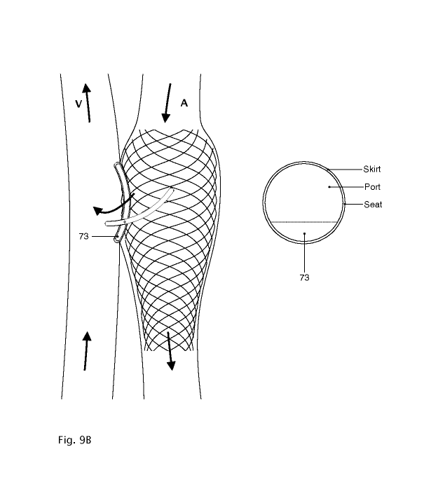

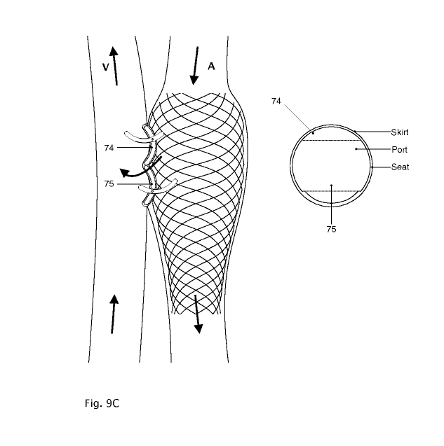

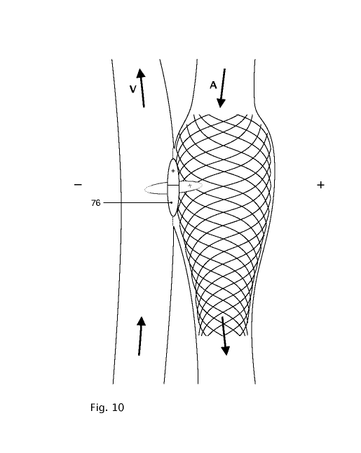

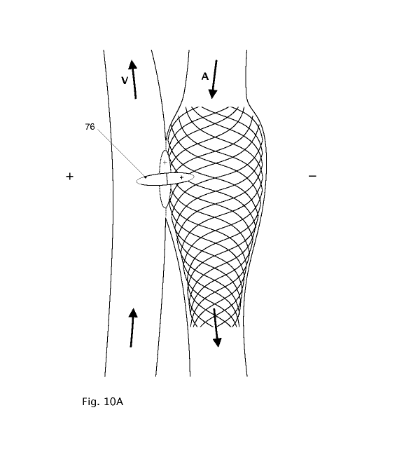

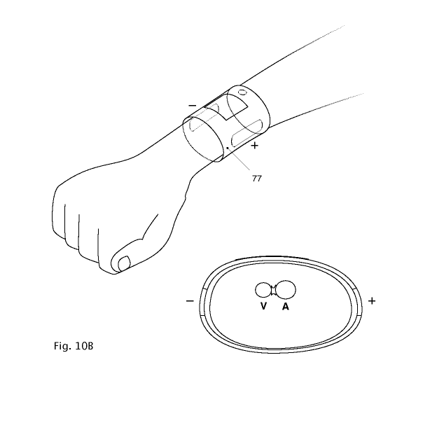

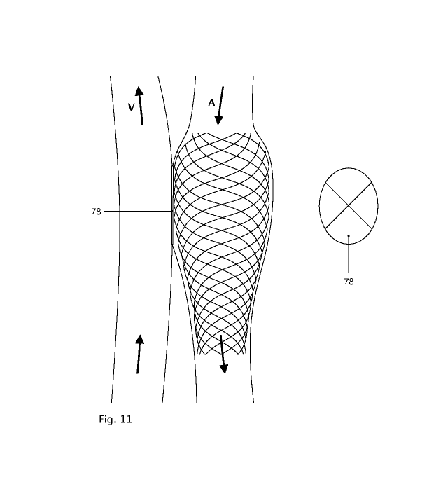

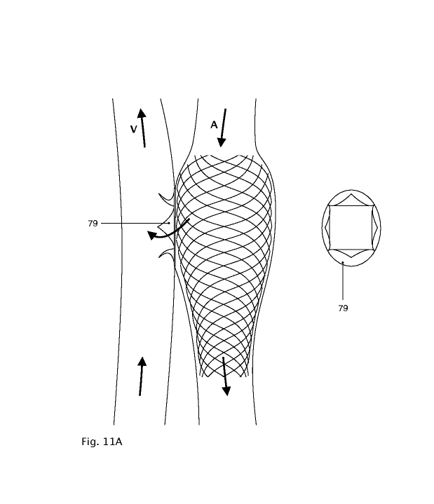

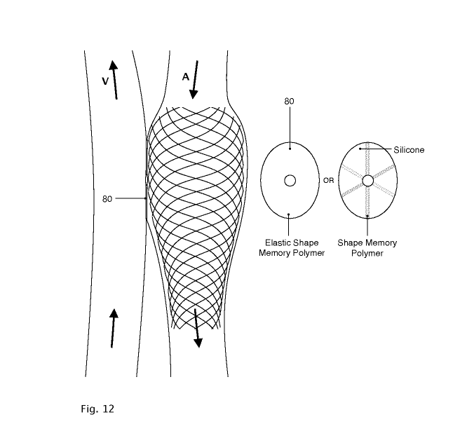

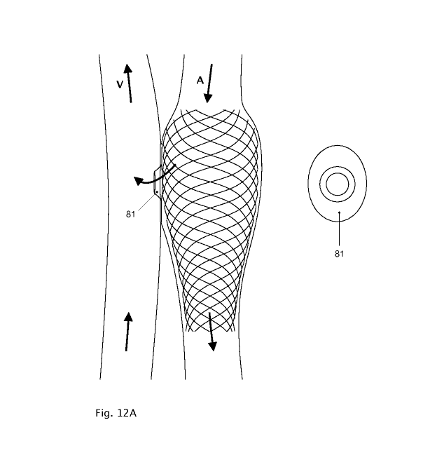

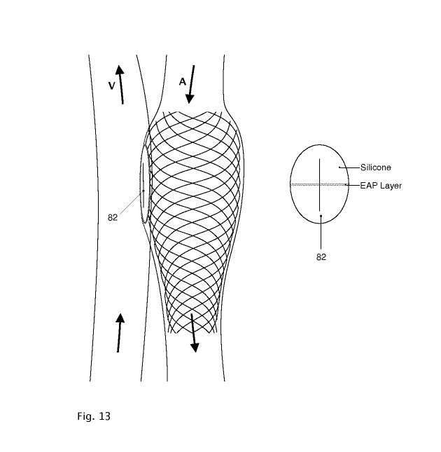

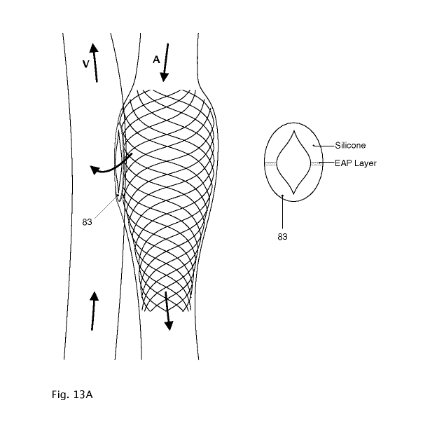

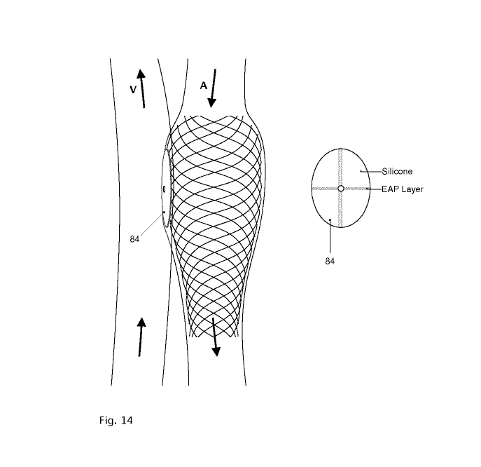

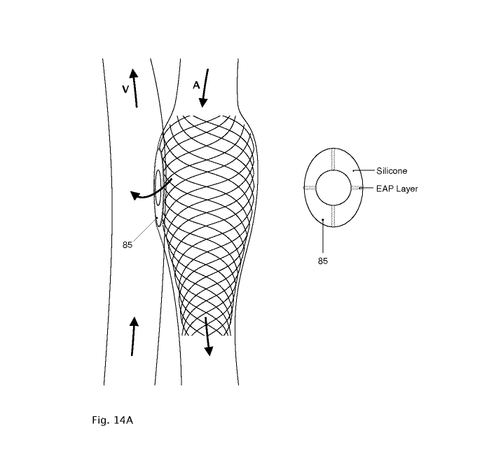

In FIG. 1, the first step is illustrated: having occluded the blood supply using a suitable haemostat, a longitudinal incision is made in the artery of a length sufficient to allow insertion of the implant. Four (perhaps more if desired) full thickness sutures 5 are made using 6/0 or 7/0 Ethalon, silk or other suitable suturing material towards each end of the incision which are used to pull each side of the incision apart as shown in FIG. 2. FIG. 3 shows a deflated balloon 1 connected to the end of a rigid T-shaped tool 2 where one part of the T extends further to one side than the other (2/3rds to 1/3^{rd}) inserted into a collapsed stent 3. The vertical stroke of the “T” passes through an aperture in the stent wall, which is surrounded by a securing ring structure 4, which is attached to the fabric of the stent 3. The stent 3 is then inserted into the artery with the longer (bottom) end inserted first. The balloon when inflated assumes a rigid, predetermined size and shape as illustrated. After the stent has been expanded within the artery, the balloon is deflated and removed. The four sutures 5 at each end of the longitudinal arterial incision are stitched through four corresponding holes in the securing ring to secure the stent structure to the artery, as shown in FIG. 4. A longitudinal incision is then made in the vein of a length corresponding to the arterial incision, as shown in FIG. 5, and the artery and vein are then sutured together around the fistula aperture as indicated in FIG. 6.

FIGS. 6A.1 to 6A.13 illustrate an alternative procedure using an AV Valve support ring with dissolvable darts and deployment mechanism as described below, to enable a defined anastomosis and provide housing for an actuatable valve.

FIG. 6A.1 shows a novel Arteriovenous fistula valve ring 10 (AV ring), designed to enhance the surgical procedure of creating an arteriovenous fistula of defined size and shape while also providing housing for an actuatable valve of the type described below, where the main purpose of the valve is to regulate blood flow between the artery and the vein. The ring 10 has four elastic arms 11 which may be bent from the rest position shown on the right in FIG. 6A.1 to a bent down position as shown on the left of that Figure.

Bending down is effected by means of a sleeve 15 forming part of an insertion and placement tool. The ring 10 is held in the tool by a pair of spring arms 12 which have tabs at their lower ends which fit into slots 13 at the sides of the ring 10. Slots 13 connect with a set of slots 14 extending from the top of the ring 10 as shown so as to provide a series of bars 15 integral with the ring which assist in attaching the ring 10 to the arterial and venous walls, as shown in FIG. 6A.11.

The insertion and placement tool shown in FIGS. 6A.2 and 6A.4 to 6A.9 constitutes a hand-held delivery device enabling the surgeon to insert the ring into the artery which has been prepared as described above, i.e. by occluding the blood flow using a suitable haemostat and making a longitudinal incision as shown in FIG. 6A.3.

The insertion and placement tool is preloaded with the ring 10 which is pulled back into the sleeve 11 so as to deflect the four arms 11 to align in the direction of a central sleeve 16 which extends upwardly as shown in FIG. 6A.2, and which surrounds a shaft 17 (shown in FIGS. 6A.4, 6A.6 and 6A.8).

Shaft 17 is connected to a transverse base plate 18 (shown in FIGS. 6A.5, 6A.7 and 6A.9), while sleeve 16 is fixed to sleeve 15.

Located within sleeve 15 is a compressed annular element having four outwardly projecting arms 20, each of which carries a downwardly projecting barb 21 which is a friction fit in a blind downwardly open bore in the end of each arm 20. Each barb 21 has a head which fits in the blind hole, and which is wider than the shaft of the barb 21.

Once the ring has been placed within the artery, sleeve 15 is withdrawn sufficiently first to enable arms 11 to spread outwards to lie against the inner wall of the artery, and then to allow the projecting arms 20 to spring outwardly as shown in FIG. 6A.5 to a position in which each barb 21 lies registered with the respective hole in arm 11. Sleeve 15 is then pushed towards ring 10 (which is held against movement by sprung arms 12) so that the barbs penetrate the artery wall and then pass through the holes in arms 11. Withdrawal of sleeve 15 then leaves the barbs in place with their heads against the artery outer wall, and further withdrawal allows the spring arms 12 to part, so enabling the insertion tool to be removed entirely as shown in FIG. 6A.9, leaving the ring in place in the artery as shown in that Figure, and FIG. 6A.10.

A set of sutures 25 is now threaded into the ring 10 as shown in FIG. 6A.11 and through the artery and vein walls as shown in FIG. 6A.12 (which shows, for the sake of clarity, only two of the eight sutures which are actually used in practice). When the entire set of sutures are tightened and tied off, the vein and artery are sutured together as shown in FIG. 6A.13.

The darts 16 are dissolvable surgical darts, which act as temporary sutures holding the AV ring in place once the insertion has been used by the surgeon to press the darts through the artery (or vein) wall and to connect with the legs 11 located inside the artery, prior to the artery and vein and the AV ring being sutured together, whereafter, as the normal healing takes place, the darts 16 simply dissolve.

FIGS. 6B.1 to 6B.8 illustrate an alternative embodiment of the AV ring, designed, again, to enhance the surgical procedure of creating an arteriovenous fistula and to house a valve to regulate blood flow between the artery and the vein. In this instance the temporary support architecture which dissolves completely leaving only the AV Ring and the necessary sutures, consists of sprung dissolvable arms. Other aspects are as described with reference to FIGS. 6A.1 to 6A.12, and the same reference numbers are used.

As in the case of the embodiment shown in FIGS. 6A.1 to 6A.12, the AV ring denoted 30 in FIGS. 6B.1 to 6B.6 may also be used independent of an actuatable valve where the main purpose is to create an anastomosis of defined size. In contrast to the previous embodiment, the periphery of the ring 30 carries four pairs of springy arms 31 made of material which, once the ring is sutured in place, dissolve over time.

As in the use of the previous embodiment, having occluded the blood flow using a suitable haemostat, a longitudinal incision is made in the artery, and an AV ring 30 is placed in the artery (or possibly the vein). The ring is placed using a hand held delivery device as described earlier which locates the ring with the eight (or more) sprung dissolvable arms 31 which, when sleeve 15 is withdrawn, grip the artery wall both inside and outside, stabilising the AV ring position. This assists the surgeon in holding the ring in position relative to the surgical incision. This has the advantage of allowing the surgeon to accurately locate and maintain position of the AV ring prior to the artery and vein being sutured together. The sprung arms 31 dissolve after surgery, leaving the AV ring 30, the artery and the vein sutured together.

The artery and vein and the AV ring are sutured together, pushing the sutures through the specially designed edge detail on the ring. The supporting arms 31 dissolve after surgery to leave an anastomosis of fixed dimension, and which may house an actuatable valve.

### Detailed Disclosure 2

### Novel Stent Design [FIG. 7]

FIG. 7 shows three novel stent designs in accordance with the invention. Each stent is designed for insertion into the arterial side of an arteriovenous fistula, and provides for significant advantages over a standard native fistula. The stents are shown in their expanded state. The stents may be made of Nitinol or another suitable, biocompatible material, a shape memory polymer or combination of such polymers, a shape memory alloy or combination of such alloys. The structure of the stents allows for an aperture to be constantly and reliably open (preventing collapse). Second, the structure allows for an aperture of predetermined size to be maintained, allowing a flow of blood sufficient for effective dialysis (usually >300 ml/min to achieve a Kt/V value of approximately 1.2) while not so great as to cause an unnecessary degree of “steal” or cardiac haemodynamic burden. The radius of the desired aperture (denoted RA in FIG. 7) may be calculated where RA is inversely proportional to the blood pressures and internal diameters of the artery and the vein. The resulting blood flow through the fistula is also dependent upon the state of the vessels in a given patient and the resulting distal pressure in the artery, and also the angle of the vein relative to the artery (in side to end fistulas).

The aperture is set approximately two thirds of the way towards the top of the stent, allowing a longer, tapering tail to manage shear stresses caused by arterial blood flow more easily and effectively.

Each of the stent structures shown in FIG. 7 includes an apertured flange which allows the shape and size of the aperture to be maintained as well as secured to the artery. These flanges may be in the form of a continuous ring 40, as illustrated in section 1 of FIG. 7, individual eye holes as illustrated in section 2 of FIG. 7 where they are proud of the stent wall and denoted 41 or they may be flush with the artery wall denoted 42. The stent may have a guiding “scoop” 43 as shown diagrammatically in FIG. 7, part 3 which is located in use either within the arterial side or extending into the vein, or both, as illustrated in section 3 of FIG. 7, to guide blood flow and reduce turbulence and shear stress.

The shape of the structure, wider at the top and narrower at the bottom, causes a drop in flow velocity above the fistula aperture, reducing the stress effects of the arterial pressure changes which naturally occur as blood is pumped through the vessel, causing peristaltic bulges along its length. The “shoulders” of the stent extend in such a way as to minimise the likelihood of shearing and separation between the artery wall (endothelium) and the stent. This is important, since this separation can lead to infection, the build-up of plaques and stenosis. In this regard, the stent may be coated with a chemical material such as polyphosphazine, which allows the release of agents to combat thrombosis, clotting or stenosis.

Fistula Insert Capable of Receiving an Actuatable Valve for an Arteriovenous Fistula

FIG. 8 shows at the top left, diagrammatically, a ring 50 which may be of a size chosen to provide a predictable anastomosis and the insertion of which prevents vein collapse during the maturation of the fistula. Ring 50 is preferably provided with a double skirt arrangement 51, 52. An actuatable valve, as described below, may be fitted to ring 50 before insertion.

The stent or double skirt structures illustrated in FIG. 8 and described above could house an actuatable valve, allowing the blood flow to be further regulated so that increased venous blood pressure (sufficient to allow for effective dialysis) was only present while the fistula was maturing (usually 3 to 6 months after first being created) and, thereafter, only during dialysis.

Several embodiments of the invention are now explained. The scope of the invention is not limited to the particular parts shown which are meant for illustrative purposes. Well-known circuits in the electrically operated embodiments, for example, have not been drawn so as not to obscure the understanding of the valve mechanism.

Referring to FIGS. 8 to 18 of the Drawings:

FIG. 8 shows a flange structure extending into the artery and the vein should a stent structure not be required or desirable. The example shown shows a simple flap valve hinged toward the bottom of the aperture seat and extending into the vein. The flange structure may employ any of the materials suitable for the stent device, and may be a mesh (as would be normal for a stent) rather than the solid structure illustrated.

FIG. 8A shows diagrammatically by way of illustration how an implant of the type discussed above could be located and could operate. The figure shows a “side-to-side” fistula, but an “end-to-side” fistula would equally accommodate such an implant, as shown in FIG. 8B. FIG. 8A shows a simple flap mechanism for the valve, the flap being a disc or ellipse shape hinged horizontally across the fistula aperture, with the upper section on the venous side and lower section of the arterial side made of a magnetic material. A spring (not shown) will normally hold the flap open or partially open until a magnetic force is applied from both sides of the valve to open the valve fully, or close it fully depending on the orientation of the magnetic field.

As can be seen on the left-hand side of the Figure, the radial artery shown on the right and the cephalic vein on the left are shown drawn together and a fistula is created between them enabling blood to flow from the radial artery into the cephalic vein as shown by the J-shaped arrow on the left-hand side of FIG. 8A. FIG. 8A shows a side-to-side configuration and FIG. 8B shows a side to end configuration. The fistula is normally simply an opening between the radial artery and the cephalic vein and because of the blood which flows through that opening, there is a reduced arterial blood flow going e.g. to the hand of the patient (at the bottom right of the diagram) and it is at a reduced pressure compared to normal. The venous blood flow is increased.

When dialysis is to be carried out, two needles 60, 61 are inserted into the cephalic vein, the upstream one 60 taking blood to the dialysis machine and the downstream one 61 for the blood flowing from the dialysis machine into the cephalic vein.

In accordance with the invention, as shown on the enlarged section on the right in FIGS. 8A and 8B, there is a diagrammatic simple valve consisting of a disc which, in the position shown in full lines, seals the aperture between the two vessels and which can be rotated during dialysis to the position shown in dashed lines enabling blood to flow directly through from the radial artery into the cephalic vein from which it is promptly removed by the upstream blood removal needle 60 inserted into the cephalic vein.

FIGS. 9 and 9A show a possible valve structure for providing an arteriovenous fistula which can be opened at will. There are three main elements: the port 70 (the moving structure controlling blood flow); the seat (the structure enclosing the port and, with the port, comprising the valve); and the skirt (the structure securing the valve in place).

The valve port 70 is comprised of round disc which pivots within a ring. The disc rotates to control the flow of fluid through the port. The port is closed when the meeting surfaces/edges of the disc and ring align and the port is open when these surfaces/edges are not aligned. The port is opened through the rotation of the disc about the pivotal axis.

In the case of the arteriovenous fistula valve, the axis about which the disc can rotate is set to one side so that the disc can be held open by the differential pressure between the blood in the artery and that in the vein and the resulting flow of the blood from the artery into the vein, much like a flap valve.

A magnet can be included in the disc such that when a magnetic/electro-magnetic field is positioned near the valve the port 70 opens, closes, or is held open or closed.

When dialysis is to be carried out, a magnetic field is applied to open the valve. The valve can then be held open during dialysis either by means of the blood flow pressure differential or by continued operation of the magnetic field on the port magnet. Continuous use of the magnetic field during dialysis means that the port magnet will put a continuous load on the system, but this may be acceptable. However there may be benefits in having the valve only open when in the presence of a magnetic field.

The magnetic field can also actuate the port to close or partially close the valve. It might be beneficial for the valve to require a magnetic field to hold the port closed This would mean that no specialist equipment would be required for blood access for the purposes of dialysis.

The seat which may be in the form of a ring as described above provides structural support for the pivot axis and the corresponding surfaces that meet to close the port and control the flow of fluid/blood.

The seat may be set in a skirt made of a material that is compatible with the tissue in which the valve is designed to operate (stenting material such as Nitinol). The valve seat can have a single skirt which can also be designed to act as a stent, i.e. to reinforce the vein or artery wall adjacent the valve as well as providing the physical connection between the tissue and valve. This connection should provide a seal between the valve and the tissue.

A single skirt can also align between two tissues so that the valve controls flow between two isolated areas such as an artery and vein. The skirt can be joined just to one surface of tissue. Alternatively a double skirt can be provided which acts as a connection and partial stent for two areas of tissue (artery and vein), e.g. the double skirt 51, 52 as shown in FIG. 8. The skirt material should promote the sealing and long-term structure support for the valve in the tissue.

FIG. 9 shows a single hinged valve opening towards the bottom of the valve seat and which, when the valve is opened, extends into the vein. The valve flap 70 is curved to reduce turbulence through the anastomosis. It might be desirable to have two flaps, as shown in FIG. 9, denoted 71 and 72, which are curved in opposing directions and hinged toward the top and bottom of the valve seat. Such an arrangement reduces the extent to which the flaps 71, 72 extend into each of the artery and vein, thus reducing turbulence and occlusion of blood flow either side of the anastomosis. FIGS. 9B and 9C show similar possible arrangements for valve mechanisms, in the former case a disc 73 which opens into the artery, and in the latter case two discs 74, 75 opening respectively into the vein and the artery. Each flap has a curved profile to encourage blood flow with minimal turbulence.

In addition to a simple magnetic mechanism, the valve could also be actuated using other mechanisms, for example a shape memory polymer or combination of such polymers, or shape memory alloy or combination of such alloys, or a combination of polymer and alloy. Actuation could be effected by heat, light or electromagnetic charge to cause the material to reversibly assume its transition state. The following drawings show some possible approaches diagrammatically:

FIGS. 10 and 10A show a simple, magnetically actuated pivoting disc or ellipse 76. The top part (usually about half) of the disc is of permanently magnetised material while the lower part is either magnetic but not permanently magnetised material, or is permanently magnetised but with opposite polarity to the top part . The valve is housed in Nitinol stenting which has a very low relative magnetic permeability (greater than 1.002). When positive and negative poles are situated on opposite sides of the valve, the disc will either swing open as shown in FIG. 10A or closed as shown in FIG. 10, depending on the orientation of the poles.

FIG. 10B shows an arm- or wristband 77 that is permanently magnetised, or contains magnets, with positive and negative poles as indicated. Worn one way, the valve will be open and when reversed the valve will be closed. The orientation of the wristband relative to the vein and artery in the arm are shown in the diagrammatic section on the lower right in FIG. 10B.

The arm- or wristband 77 can also contain an integrated OLED or LED light source, tuned to the far infra-red (FIR) spectrum. FIR therapy has been shown to combat stenosis in arteriovenous fistulae. In addition, the arm- or wristband 77 may house a sensor which can monitor blood flow through the fistula, sending the information to a smart phone, for example.

FIGS. 11 and 11A show a possible embodiment of the valve utilising electroactive polymeric material 78. A surface acoustic wave device (SAW), interdigital transducer (IDT) and an antenna can be housed in the skirt of the valve, allowing the valve to be operated remotely through radio frequency (RF) pulses, stiffening or relaxing the polymeric material to close the valve, FIG. 11, or as shown at 79 in FIG. 11A, open the valve. Such operation has the distinct advantage of not requiring an energy source within the implant itself. It is evident that shape memory polymers or shape memory alloys, thermally, electromagnetically or light activated might equally be employed with similar effect.

FIGS. 12 and 12A show another possible embodiment for the valve, employing a valve disc 80 of shape memory polymer or alloy, remotely actuated using heat, light, electro-magnetic or piezo-resistive induction. As illustrated, such material may or may not be housed within a suitably elastic material such as silicone should it not itself provide the requisite degree of flex/stretch. The disc 80 is shown with the valve closed (save for a small central aperture) in FIG. 12, while FIG. 12A shows the disc, denoted 81, when actuated to open the valve to allow blood to pass from artery to vein during dialysis.

FIGS. 13 and 13A show another possible embodiment of the valve mechanism, whereby a disc 82 of suitably flexible material such as silicone is associated with an electrically actuatable polymer, for example an aerogel (or carbon nanotube microfiber) or a shape memory polymer or alloy (heat, light or electromagnetically activated) in the form of a layer of fibres denoted EAP lower in the drawing. A simple slit in the flexible material may be pulled open by the contraction of the fibres on each side of the slit, as illustrated at 83 in FIG. 13A when the electrically actuatable polymer is actuated.

FIGS. 14 and 14A show a valve mechanism similar to that in FIGS. 13 and 13A, but with a hole on the flexible material 84 pulled apart on contraction of surrounding actuatable fibres 85 in FIG. 14A, thus forming a sphincter-like arrangement.

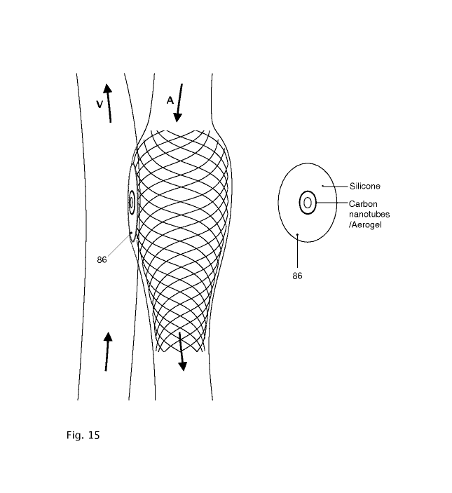

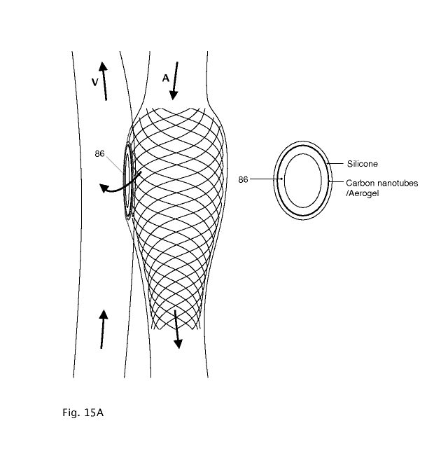

FIGS. 15 and 15A show an embodiment of the valve utilising carbon nanotube “aerogel” fibres whereby a circular strand of this material is placed within a suitably elastic and biocompatible material (e.g. a silicone disc 86) to form an artificial sphincter muscle. The valve requires a battery to activate the muscle, housed within the stenting material. When a small voltage is applied to the carbon nanotube aerogel it relaxes or constricts just as a circular muscle would relax or constrict to open and close the valve/sphincter accordingly. Such aerogels can operate at virtually any temperature (0-1,600° K), they constrict over 1000 times more quickly than natural muscle. Natural muscle typically constricts to a degree of 20% to 40%, whereas an aerogel can constrict up to 200% making the material extremely efficient. In general, use of an aerogel is preferable to a shape memory polymer, which is in turn preferable to a shape memory alloy, since contraction efficiencies descend in that order.

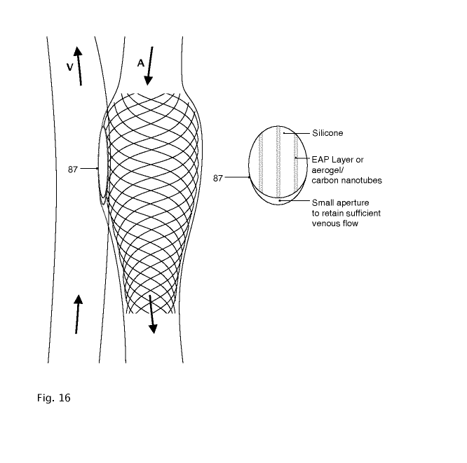

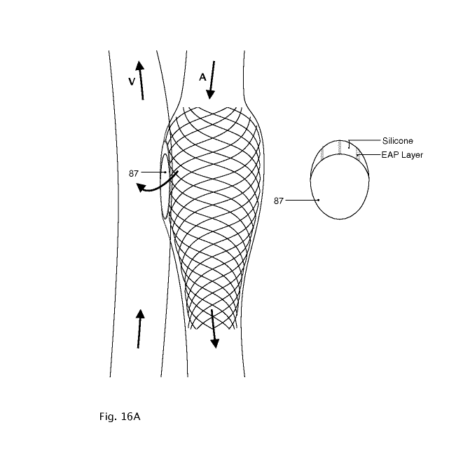

FIGS. 16 and 16A show a curtain-type structure employing similar possible actuation mechanisms as in the structure illustrated in FIGS. 15 and 15A, wherein the memory shape material or carbon nanotube structure is contained within a flexible material 87 affixed to the top portion of a skirt and acts to pull the flexible material upward, increasing the size of the aperture accordingly 87 in FIG. 16A. The structure, as illustrated, maintains a permanent small aperture towards the bottom of the port so that slightly increased venous blood flow may be maintained.

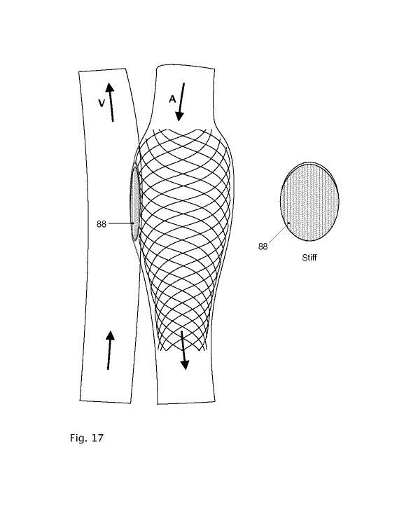

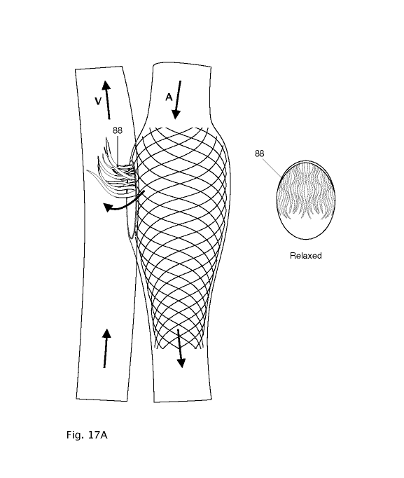

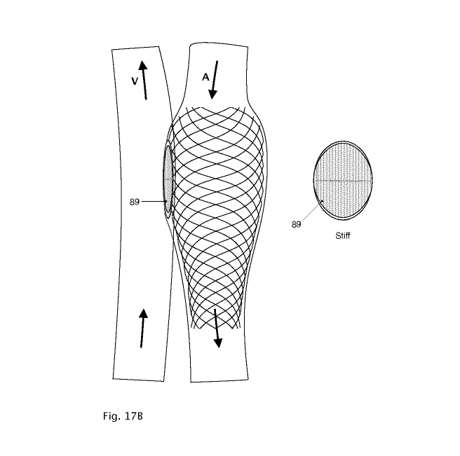

FIGS. 17 and 17A employ a polymer that may be reversibly made rigid or flexible, arranged in vertical sheets 88 extending down from the upper part of the skirt. While rigid, the sheets effectively occlude the anastomosis as shown in FIG. 17, but when flexible they allow blood to flow through the fistula forming streamers 89, as illustrated in FIG. 17A.

FIG. 18 shows an alternative form of actuatable valve which operates in a fashion analogous to a camera lens stop.

The valve consists essentially of a base member 95 having a central aperture and a central disc-shaped depression in one face. A toroidal ‘Donut’ of elastic material, for example a silicone rubbery ring 94, sits in the depression, and a spacer disc 93 with a central aperture is located between ring 94 and an electroactive polymer “muscle” ring 92. Ring 92 is sutured to a ring 91 mounted on the side of a stent 90.

The valve may be actuated e.g. by providing a standing acoustic wave antenna coupled to the muscle ring 92. When stimulated by sonic irradiation, ring 92 contracts so that ring 94 is compressed and seals the passage through the valve, as shown at the bottom of the two diagrams on the right in FIG. 18. The sonic irradiation may be provided via an arm- or wristband worn by the patient, and capable of being switched off when a dialysis session is to be carried out so as to enable blood to flow from artery to vein as desired. By varying the degree of sonic irradiation, the extent to which the valve is open may be precisely controlled.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A stent structure for use in surgery, the structure comprising an expandable stent body having an inlet and an outlet end and characterized by an aperture in the wall of the stent intermediate its ends and adapted to enable liquid flowing through the stent to pass from or into the vein or artery in which the stent is deployed.

31. The stent structure according to claim 30 in which the aperture is provided with a mounting ring fixed to the stent body and enabling the stent to be fixed around the ring to an aperture in the wall of a vein or artery.

32. The stent structure according to claim 30 in combination with a mounting ring having a plurality of radial arms which can be elastically deformed to lie in the direction of the axis of the ring to enable them to pass through the aperture in the wall of the stent.

33. The stent structure according to claim 32 wherein the radially extending arms are adapted to be temporarily fixed to the wall of a vein or artery.

34. The stent structure according to claim 33 wherein each of the arms has an aperture at its distal end through which a headed hook or dart, having a head larger than the aperture at the distal end of the arm, may be driven to penetrate the wall of a vein or artery to hold the end of the arm in a fixed position on the vein or artery wall.

35. The stent structure according to claim 30 in combination with a mounting ring having a plurality of elastic clipping structures about its periphery which may be elastically deformed by a deforming force to enable the insertion of the ring into the aperture in the stent structure to occur and which clipping structures spring back on removal of the deforming force to grip the material of the stent and hold the mounting ring relative thereto.

36. The stent structure according to claim 34 wherein the headed hooks or darts, or the elastic clipping structures respectively are formed of a material which will in time dissolve in the bloodstream.

37. The stent structure according to claim 30 wherein the aperture in the stent wall is provided with a valve structure enabling the cross-section of the aperture through which liquid may flow to be varied.

38. The stent structure according to claim 37 wherein the valve structure enables the flow through the aperture to be cut off.

39. The stent structure according to claim 37 wherein the valve structure includes one or more occluding members movable from a position in which liquid can flow through the aperture to a position in which the one or more occluding members substantially or wholly block liquid movement through the aperture.

40. The stent structure according to claim 39 wherein the position or frigidity of the one or more occluding members may be changed by the application of a magnetic, sonic or electric field or light to the occluding member.

41. The stent structure according to claim 40 wherein the one or more occluding members is formed of or includes portions of a piezoelectric material which when subjected to an electrical potential change the size of the flow path through the aperture.

42. The stent structure according to claim 39 wherein the one or more occluding members is urged by spring means towards a closed or open position.

43. The stent structure according to claim 39 wherein the one or more occluding members is elastic and is biased to a position in which the aperture is wholly or nearly closed.

44. The stent structure according to claim 30 wherein the aperture in the stent wall is closer to one end of the stent than the other and the stent wall is constructed so that when expanded by inflation of a balloon located within the stent the lateral expansion of the stent is greater towards the end of the stent closer to the aperture.

45. A valve unit for use in the control of blood flow through an arteriovenous fistula which comprises a ring of biocompatible material adapted to be attached at its periphery to incisions made in an artery and a vein and, located bridging the ring, a flow restricting or flow occluding member which may be actuated to vary the size of the aperture in the ring.

46. The valve unit according to claim 45 and having attached to its periphery at least one skirt or flange adapted to be attached to the edges of an incision made in the wall of an artery or vein, or to an aperture in a stent wall.

47. The valve unit according to claim 45 wherein the flow restricting or occluding member is one or more pivotally mounted flaps.

48. The valve unit according to claim 45 wherein the flow restricting or occluding member is an elastic membrane associated with means to expand or enable contraction of an aperture in the membrane.

49. The valve unit according to claim 45 wherein the flow restricting or occluding member is an array of strips or fibres which, when rigid, restrict flow through the aperture and which may be rendered flexible to enable flow through the aperture.

50. An assembly of a mounting ring and insertion tool for use in locating a mounting ring in an aperture wherein the mounting ring has a plurality of outwardly facing slots and the insertion tool has a pair of jaws with ends engageable in the slots to hold the ring between them, and a slidable sleeve set around the jaws and having an internal shape corresponding to the shape of the ring and movable to a position in which the ring is held wholly within the sleeve, and wherein the mounting ring has a plurality of elastic members attached to its periphery which may be elastically deformed to enable the ring to be inserted in the sleeve and which, when the ring is released from the sleeve, spring out to extend beyond the periphery of the ring.

51. The assembly according to claim 50 wherein the elastic members form clipping pins capable when released from the confines of the sleeve of gripping the edges of an aperture into which the sleeve has been inserted.

52. The assembly according to claim 50 wherein the insertion tool contains located within the sleeve and adjacent the mounting ring, a plurality of radially outwardly biased arms which, when the sleeve is moved to release the arms, move pins at or near their ends into a position registered with the position of a plurality of elastic members attached to the periphery of the ring, and wherein movement of the sleeve drives the pins into engagement with the elastic members attached to the periphery of the ring.

53. A surgical procedure for establishing an arteriovenous fistula between an artery and a vein enabling blood to flow directly from the artery into the vein wherein apertures in the walls of artery and vein are made, and the edges of the apertures are fixed to a valve member which can be selectively opened or closed to increase or decrease or cut off blood flow from the artery into the vein.

54. The surgical procedure according to claim 53 wherein a stent structure according to claims 30 is inserted in a collapsed state into the vein or artery and then expanded to lodge securely in the vein or artery with the aperture aligned with the apertures made in the artery and vein walls.

55. The surgical procedure according to claim 54 wherein the valve member is initially located in position relative to the aperture in the vein or artery into which the stent is inserted by means of a plurality of temporary fixing means which, over time, dissolve in situ, and where the valve member is, while such fixing means are in place, connected to the edges of incisions made in the vein and artery.

56. The surgical procedure according to claim 55 wherein the edges of the apertures in the walls of the vein and artery are affixed to the valve member or to the stent structure surrounding the valve structure by suturing.

57. A surgical procedure for creating an arteriovenous fistula wherein, prior to connecting the edges of incisions made in the vein and artery to create the fistula, a stent according to claim 30 is inserted in the artery and expanded with the aperture in the stent wall registered with the incision in the artery.

58. The surgical procedure according to claim 57 wherein the expansion of the stent is greater towards the upstream end of the stent than towards the downstream end.
